(19) United States
(12) Patent Application Publication
(10) Pub. No.: US 2016/0213788 A1
(43) Pub. Date: Jul. 28, 2016

(54) ACTIVE TARGETING ANTITUMOR DRUG AND PREPARATION METHOD THEREFOR

(71) Applicant: NINGBO INSTITUTE OF MATERIALS TECHNOLOGY & ENGINEERING, CHINESE ACADEMY OF SCIENCES, Ningbo, Zhejiang (CN)

(72) Inventors: Juan LI, Ningbo, Zhejiang (CN); Aiguo WU, Ningbo, Zhejiang (CN)

(21) Appl. No.: 14/917,256

(22) PCT Filed: Apr. 18, 2014
(86) PCT No.: PCT/CN2014/075727

§ 371 Date: Mar. 7, 2016

(30) Foreign Application Priority Data
Sep. 9, 2013 (CN) 201310407938.0

(51) Int. Cl. A61K 47/48

(52) U.S. Cl. CPC A61K 47/48238; A61K 47/48884; A61K 47/48284

(57) ABSTRACT

Disclosed is a composite. The composite comprises a nanocarrier and targeting molecules coupled to a surface of the nanocarrier. The particle size of the nanocarrier is smaller than 200 mn, and the polydispersity index (PDI) is smaller than 0.5. Also disclosed are an antitumor medicament comprising the composite and antitumor drug carried in the nanocarrier of composite, a preparation method therefor, and uses thereof. The composite can convey the antitumor drug into a tumor cell, and has a strong killing effect to the tumor cell.

### TECHNICAL FIELD

The present invention relates to the field of pharmaceutical technology, and particularly to an active targeting antitumor drug and the preparation method therefor.

### BACKGROUND

An active targeting drug is a kind of drugs or drug carriers whose surface is modified by an active targeting molecule (e.g., antibody or ligand, etc.), so it can bind with specific antigens or receptors of certain tissues or cells, thereby achieving a function of targeting specific cells and tissues. Due to the high specificity, high selectivity and high affinity between antigen and antibody or between receptor and ligand, active targeting has a higher targeting efficiency than passive targeting, so that the study of active targeting medicine delivery system is very active at home and abroad.

The active targeting drug, which is designed based on the principle of specific binding between an antigen and an antibody, still has various problems such as low effective concentration of targeted drugs, strong racial specificity, high immunogenicity and high R&D costs.

However, the active targeting drug, which is designed based on the principle of specific binding between ligand and receptor, has the features of high selectivity, no racial specificity, no immunogenicity, high stability as well as low cost, thus becoming the hotspot and focus in current tumor targeting delivery system design, including researches on tumor-targeting medicine mediated by folate receptors, transferrin receptors, integrin receptors and polypeptide receptors. In recent years, the tumor-targeting medicine mediated by polypeptide receptor has attracted more and more attention.

But so far, the study on anti-cancer drug targeting delivery system mainly aims at targeting to the tumor tissue. In recent years, how to deliver more drugs into tumor cells after drugs reach tumor site so as to achieve a targeting delivery of medicine into tumor cells, has become the research focus of targeting medicine delivery system. Many pharmaceutical delivery systems exhibit a relatively good tumor targeting, and they can deliver drugs to surface of tumor cells. However, after the carrier binds with targeted cell, because the carrier has a weak ability of entering into the targeted cell, most of the drugs are released outside the targeted cell. Further, when the drugs effect, they also activate various genes of the tumor cell membrane so that various molecular mechanisms are synergistically involved in process of formation of resistance phenotype. Once the drug resistance is established, it further reduces the drug efficiency of entering into tumor cells so as to result in an extremely low intracellular drug concentration. Therefore, the growth of the tumor cell can not be effectively inhibited.

Neuropeptide Y (NPY) is a kind of hormones widespread in the central nervous system and peripheral nervous system and maintains homeostasis. Six kinds of NPY receptors have been found and identified, namely NPY Y₁, Y₂, Y₃, Y₄, Y₅ and Y₆, which are widespread in the central nervous system and peripheral nervous system in mammals. The function of NPY is inextricably linked to its receptors, in other words, the diversity of receptor causes the function diversity of NPY. Studies have shown that the known drugs for neuropeptide receptor are mainly used for treating diseases related to physiological disorder, including obesity, cardiovascular disease, high cholesterol, epilepsy, anxiety and the like. However, anti-tumor drugs with NPY receptors as target are rare, and especially no anti-tumor drugs for treating kidney cancer, stomach cancer, breast cancer and ovarian cancer have been reported.

Agonists and inhibitors based on different NPY receptors (i.e. the ligands of the receptor) have been widely studied, but it is still difficult to choose one suitable ligand to modify pharmaceutical carrier due to the diversity as well as universality of NPY receptors. Therefore, it has become a key in the design and R&D of targeted drugs to select a suitable ligand to modify pharmaceutical carrier, so that the modified pharmaceutical carrier can bind a receptor of tumor cell with high specificity without affecting biological activity of other intracellular receptors, and can directionally deliver the anti-tumor drugs in the carrier into tumor cells, thereby increasing the effective drug concentration in tumor cells.

### SUMARY OF THE INVENTION

One object of the present invention is to provide an active targeting antitumor drug and the preparation method therefor.

In the first aspect of the present invention, a composite is provided, wherein the composite comprises:

a nanocarrier; and

a targeting molecule coupled to a surface of the nanocarrier;

wherein the targeting molecule is selected from the group consisting of: [D-Arg²⁵]-NPY, [D-His²⁶]-NPY, [D-Arg²⁵, D-His²⁶]-NPY, [Arg⁶, Pro³⁴]pNPY, [Asn⁶, Pro³⁴]pNPY, [Cys⁶, Pro³⁴]pNPY, [Phe⁶, Pro³⁴]pNPY, [Arg⁷, Pro³⁴]pNPY, [D-His²⁶, Pro³⁴]NPY, [Phe⁷, Pro³⁴]pNPY, [Pro³⁰, Nle³¹, Bpa³², Leu³⁴]NPY(28-36), [Pro³⁰, Nal³², Leu³⁴]NPY(28-36), [Pro³⁰, Nle³¹, Nal³², Leu³⁴]NPY(28-36), BIBO3304, PD160170, LY366258, J-104870, LY 357897, J-115814, and combinations thereof;

and the nanocarrier has a particle size which is ≦200 nm and a polydispersity index (PDI) which is smaller than 0.5.

In another preferred embodiment, the content of targeting molecule is 1.11 to 22.2 wt % of total weight of the composite.

In another preferred embodiment, the content of targeting molecules is 5.60 to 11.1 wt % of the total weight of the composite.

In another preferred embodiment, the composite has one or more of the following characteristics:

(a) the composite binds with breast cancer cells, ovarian cancer cells, renal cancer cells or stomach cancer cells in high specificity;

(b) the particle size of the nanocarrier is 10-200 nm.

In another preferred embodiment, the nanocarrier is selected from the group consisting of: protein-based nanoparticle, oligopeptide-based nanoparticle, phospholipid-based nanoliposome, polysaccharide-based nanoparticle, polyether-based nanoparticle, polyester-based nanoparticle, and polyester-based polymeric micelle.

In another preferred embodiment, the protein-based nanoparticle is selected from human serum albumin nanoparticle, or bovine serum albumin nanoparticle.

In another preferred embodiment, the phospholipid-based nanoliposome is selected from phosphatidylcholine nanoliposome, dipalmitoyl phosphatidylcholine nanoliposome, distearoyl phosphatidylcholine nanoliposome, dipalmitoyl phosphatidylethanolamine nanoliposome, distearoyl phosphatidylethanolamine nanoliposome, or dipalmitoyl phosphatidylglycerol nanoliposome.

In another preferred embodiment, the polyester-based nanoparticle is selected from polyethylene glycol-polylactic acid nanoparticle, polyethylene glycol-polylactide glycolide nanoparticle, or polyethylene glycol-polycaprolactone nanoparticle.

In another preferred embodiment, the polysaccharide-based nanoparticle comprises chitosan nanoparticle.

In another preferred embodiment, the polyester-based polymeric micelle is selected from polyethylene glycol-polylactic acid micelle, polyethylene glycol-polycaprolactone micelle, polyethylene glycol-distearoyl phosphatidylethanolamine micelle, or polyethylene glycol-polyethyleneimine micelle.

In the second aspect of the present invention, a composition is provided, wherein the composition comprises:

the composite in the first aspect; and

an anti-tumor drug carried in the nanocarrier of the composite.

In another preferred embodiment, the anti-tumor drug is selected from the group consisting of: doxorubicin, paclitaxel, docetaxel, cisplatin, mitoxantrone, daunorubicin, vincristine, all-trans retinoic acid, pharmorubicin, lurtotecan, irinotecan, 2-methoxyestradiol, gemcitabine, vinorelbine, 5-fluorouracil, methotrexate, capecitabine, lomustine, etoposide, and combinations thereof.

In another preferred embodiment, the anti-tumor drug is embedded in the nanocarrier of the composite.

In another preferred embodiment, in the composition, the encapsulation efficiency of nanocarrier for anti-tumor drug is above 80%, and preferably 90% or more.

In another preferred embodiment, when the concentration of anti-tumor drug in the composition is 5-10 μg/mL, the killing rate of tumor cell by the composition is above 60%, and preferably above 70%.

In another preferred embodiment, the tumor cell include tumor cell of breast cancer, ovarian cancer, renal cancer, or gastric cancer.

In another preferred embodiment, the content of anti-tumor drug is 1.0 to 3.0 wt % and preferably 1.5 to 2.7 wt % of the total weight of composition.

In another preferred embodiment, the content of targeting molecule is 1.11 to 22.2 wt % and preferably 5.60 to 11.1 wt % of the total weight of composition.

In the third aspect of the present invention, a method for preparing the composition of the second aspect is provided, wherein the method comprises the following steps:

(1) providing a nanocarrier loaded with an anti-tumor drug; and

(2) coupling the nanocarrier of step (1) with a targeting molecule, thereby obtaining the composition.

In another preferred embodiment, the particle size of the nanocarrier is 200 nm or less, and preferably 10-200 nm.

In another preferred embodiment, the method for preparing the nanocarrier comprises the steps of:

(a) respectively providing an aqueous solution and an organic solution, wherein the aqueous solution comprises an anti-tumor drug and a hydrophilic membrane material, and the organic solution comprises an emulsifier;

(b) mixing the aqueous solution with the organic solution of step (a), thereby obtaining an emulsion;

(c) curing the emulsion of step (b), thereby obtaining the nanocarrier.

Or the method for preparing the nanocarrier comprises the steps of:

(a) respectively providing an aqueous solution and an organic solution, wherein the aqueous solution comprises an emulsifier, and the organic solution comprises an anti-tumor drug and a hydrophobic membrane material;

(b) mixing the aqueous solution with an organic solution of step (a), thereby obtaining an emulsion;

(c) curing the emulsion of step (b), thereby obtaining the nanocarrier.

Or the method for preparing the nanocarrier comprises the steps of:

(a) respectively providing an aqueous solution and an organic solution, wherein the aqueous solution comprises an anti-tumor drug, and the organic solution comprises a hydrophobic membrane material and an emulsifier;

(b) mixing the aqueous solution with the organic solution of step (a), thereby obtaining a first emulsion;

(c) mixing the first emulsion of step (b) with the aqueous solution comprising a dissolved emulsifier, thereby giving a second emulsion;

(d) curing the second emulsion of step (c), thereby obtaining the nanocarrier.

Or the method for preparing the nanocarrier comprises the steps of:

(a) providing a suspension which comprises an anti-tumor drug, a nanocarrier and an organic solvent;

(b) curing the suspension of step (a), thereby obtaining the nanocarrier.

In another preferred embodiment, the hydrophilic membrane material is selected from: polyethylene glycol (PEG), polyoxyethylene (PEO), polyvinyl pyrrolidone (PVP) or polyvinyl alcohol (PVA).

In another preferred embodiment, the hydrophobic membrane material is selected from: polyoxypropylene (PPO), polystyrene (PS), polyaminoacid, polylactic acid (PLA), spermine or short chain phospholipid.

In another preferred embodiment, the emulsifier is selected from: Pluronic F68, Dextran 70 or sodium cholate.

In another preferred embodiment, the coupling reaction is selected from:

(1) a condensation reaction of a carboxyl group and an amino group;

(2) an addition reaction of a sulfhydryl group and a maleimide group; or

(3) a non-covalent binding of avidin and biotin.

In the fourth aspect of the present invention, it provides a use of the composite of the first aspect for preparing a medicament for treatment of cancer.

In another preferred embodiment, the cancer comprises breast cancer, ovarian cancer, kidney cancer and stomach cancer. More preferably, the cancer comprises kidney cancer and stomach cancer.

In the fifth aspect of the present invention, it provides a use of the composition of the second aspect for preparing a medicament for treatment of cancer.

In the sixth aspect of the present invention, it provides a medicament which comprises:

the composite of the first aspect;

an anti-tumor drug carried in the nanocarrier of the composite; and

a pharmaceutically acceptable carrier.

In another preferred embodiment, the formulation of the medicament is selected from the group consisting of: solid preparations, liquid preparations and injections.

In another preferred embodiment, the administration subject of the medicament is a mammal, and preferably a human.

In another preferred embodiment, the formulation of the medicament is an injection.

In another preferred embodiment, the administration route of the injection comprises intravenous, intramuscular, subcutaneous or intracavitary administration.

In the seventh aspect of the present invention, it provides a method for treating cancer which comprises a step of administering to a subject in need thereof a safe and effective amount of a composition according to the second aspect or the medicament of the sixth aspect.

In another preferred embodiment, the cancer comprises breast cancer, ovarian cancer, kidney cancer or stomach cancer. More preferably, the cancer comprises kidney cancer or stomach cancer.

It should be understood that in the present invention, any of the technical features specifically described above and below (such as in the Examples) can be combined with each other, thereby constituting new or preferred technical solutions which will not redundantly be described one by one herein.

### DESCRIPTION OF FIGURES

FIG. 1 shows a TEM pattern and DLS particle size distribution diagram of the composition [D-Arg²⁵]-NPY-ANP-TXT.

FIG. 2 shows the changes in particle size of the composition [D-Arg²⁵]-NPY-ANP-TXT in NaCl solution, PBS solution and serum for 1-15 days.

FIG. 3 shows an comparative profile of tumor cells MCF-7 and HEC-1B-Y5 uptaking the composition [D-Arg²⁵]-NPY-ANP-TXT.

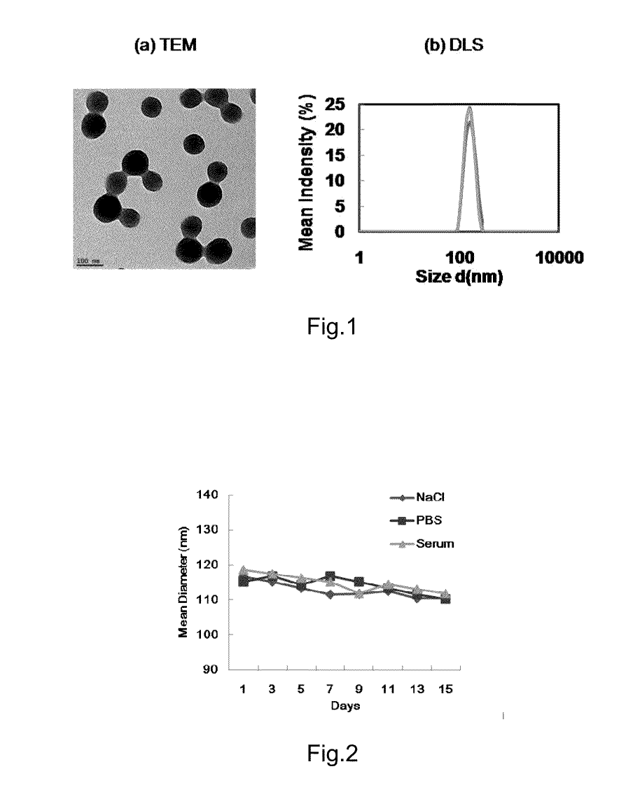

### DETAILED DESCRIPTION OF INVENTION

Through extensive and intensive long research, the inventors have unexpectedly found that a composition which is prepared by coupling certain targeting molecules with the nanocarrier loaded with anti-tumor drugs, can bind neuropeptide receptors of specific tumor cells in high specificity and directionally deliver the anti-tumor drugs into these cells, thereby increasing the effective drug concentration in tumor cells, while the composition has almost no toxic side effects on normal tissues and cells. The composition of the present invention has a strong killing effect on tumor cells, particularly on tumor cells of breast cancer, ovarian cancer, kidney cancer and stomach cancer, and therefore it is useful in preparation of a medicament for treating the above-mentioned cancers. Based on these findings, the inventors have completed the present invention.

As used herein, the term “biotin” refers to vitamin H, or called as vitamin B7 or coenzyme R, and has a molecular weight of 244.31 Da.

As used herein, the term “avidin” is a glycoprotein with a molecular weight of about 60 kDa, and mainly includes protein avidin (also called as natural avidin, ovalbumin avidin or anti-biotin), streptavidin, yolk avidin and the like.

Targeting Molecule

The targeting molecule of the present invention refers to a peptide agonist molecule or a non-peptide antagonist molecule, which can bind with neuropeptide receptors in high specificity and efficiency, thereby causing various biologically activity. The peptide agonist molecule includes (but is not limited to): [D-Arg²⁵]-NPY, [D-His²⁶]-NPY, [D-Arg²⁵,D-His²⁶]-NPY, [Arg⁶, Pro³⁴]pNPY, [Asn⁶, Pro³⁴]pNPY, [Cys⁶, Pro³⁴]pNPY, [Phe⁶, Pro³⁴]pNPY, [Arg⁷, Pro³⁴]pNPY, [D-His²⁶, Pro³⁴]NPY, [Phe⁷, Pro³⁴]pNPY, [Pro³⁰, Nle³¹, Bpa³², Leu³⁴]NPY(28-36), [Pro³⁰, Nal³², Leu³⁴]NPY(28-36) and [Pro³⁰, Nle³¹, Nal³², Leu³⁴]NPY(28-36), and the non-peptide antagonist molecule includes (but is not limited to): BIBO3304, PD160170, LY366258, J-104870, LY 357897 and J-115814.

Through research, the inventors have unexpectedly found that the above targeting molecule of the present invention can bind breast cancer, ovarian cancer, kidney cancer and gastric cancer cells in high specificity, but can not specifically bind brain tumor cell and endometrial tumor cell.

As used herein, “bind . . . in high specificity” refers to that, under the same conditions, after binding with tumor cells and non-tumor cells (i.e., normal cells) respectively, the uptake rate of the composite of the present invention satisfies the following condition: B₁/B₀≧2.5, preferably B₁/B₀≦3, and more preferably B₁/B₀≦4; wherein B₁ refers to the uptake rate of the composite by 10,000 tumor cells; Bₒ refers to the uptake rate of the composite by 10,000 normal cells.

Composite and the preparation method thereof

The composite of the present invention is a binary composite, which is composed of a biodegradable nanocarrier and targeting molecules, wherein the targeting molecules are coupled onto surface of the nanocarrier. Excessive targeting molecule may lead to precipitation or agglomeration of the composite nanoparticles, thereby increasing diameter of the composite nanoparticles (>200 nm). In the present invention, based on the total weight of the composite, the content of targeting molecules is 1.11 to 22.2% (wt %), and preferably 5.60 to 11.1% (wt %), while the remained is biodegradable nanocarriers. The composite of the present invention has a good dispersion and stability in NaCl aqueous solution, PBS aqueous solution or serum, with no precipitation or agglomeration phenomenon.

In the present invention, the nanocarrier can be selected from: protein-based nanoparticle, oligopeptide-based nanoparticle, phospholipid-based nanoliposome, polysaccharide-based nanoparticle, polyether-based nanoparticle, polyester-based nanoparticle, polyester-based polymeric micelle, or combinations thereof. The preferred are protein-based nanoparticle, phospholipid-based nanoliposome and polysaccharide-based nanoparticle.

Preferred protein-based nanoparticle comprises human serum albumin nanoparticle (HSA), bovine serum albumin nanoparticle (BSA), or combinations thereof.

Preferred phospholipid-based nanoliposome comprises phosphatidylcholine (PC) nanoliposome, dipalmitoyl phosphatidylcholine (DPPC) nanoliposome, distearoyl phosphatidylcholine (DSPC) nanoliposome, dipalmitoyl phosphatidylethanolamine(DPPE) nanoliposome, distearoyl phosphatidylethanolamine (DSPE) nanoliposome, dipalmitoyl phosphatidylglycerol (DPPG) nanoliposome, or combinations thereof.

Preferred polyester-based nanoparticle comprises polyethylene glycol-polylactic acid (PEG-PLA) nanoparticle, polyethylene glycol-polylactide glycolide(PEG-PLGA) nanoparticle, polyethylene glycol-polycaprolactone (PEG-PCL) nanoparticle, or combinations thereof.

Preferred polysaccharide-based nanoparticle comprises chitosan nanoparticle.

Preferred polyester-based polymeric micelle comprises polyethylene glycol-polylactic acid (PEG-PLA) micelle, polyethylene glycol-polycaprolactone (PEG-PCL) micelle, polyethylene glycol-distearoyl phosphatidylethanolamine (PEG-DSPE) micelle, polyethylene glycol-polyethyleneimine (PEG-cl-PEI) micelle, or combinations thereof.

The method for preparing the composite of the invention mainly comprises the steps of: (1) preparing the nanocarriers and (2) reacting the nanocarriers with targeting molecules. The preparation method of the nanocarrier may be methods well-known to the skilled in the art, and the reactions of the nanocarrier and the targeting molecule may carry out via chemical coupling method.

One preferred coupling method is as follows:

when the nanocarriers contain carboxyl groups (such as poly-glutamic acid, poly-aspartic acid, peptides and proteins containing glutamic acid and aspartic acid, and polysaccharide having carboxyl group), one can select the targeting molecule containing an amino group, and activate carboxyl groups on the surface of the nanocarriers with EDAC and NHS (N-hydroxysuccinimide), and then add dropwise a solution of the targeting molecule having terminal amino groups, to introduce a covalent reaction between the activated carboxyl groups and amino groups of the targeting molecule, thereby forming a stable amide bond; when the nanocarriers contain amino groups (such as poly-lysine, histidine, poly-arginine, peptides and proteins containing lysine, arginine and histidine, and amino-containing polysaccharide), one can select carboxyl groups-containing targeting molecule, and then activate carboxyl groups of the targeting molecule using the above method to make the activated carboxyl groups to be grafted onto surface of the nanocarriers; as to the biodegradable nanocarriers containing neither carboxyl groups nor amino groups (such as polyether and polyesters polymer), copolymerization method can be used to make the biodegradable nanocarriers contain amino or carboxyl groups, and after forming into nanocarriers, the same method as above can be used to graft the targeting molecules onto surface of the nanocarriers.

The other preferred coupling method is as follows:

mercapto groups are introduced into the targeting molecule, and maleimide groups are introduced onto the surface of the drug loading system, and then an addition reaction in a neutral or alkaline aqueous environment (e.g. phosphate buffer) at room temperature is carried out.

Thiolated targeting molecules are mainly prepared via the reaction between sulfhydryl reagents (such as 2-IT, SPDP, SATP and SSDD etc.) and amino groups of the targeting molecules;

In order to introduce maleimide into the drug loading system, lipid molecules and maleimide (MAL) modified polymer materials (such as MAL-PEG-PLA, MAL-PEG-PLGA, MAL-PEG-PCL, MAL-PEG-DSPE) are mixed and dissolved in an organic solvent, and via filming hydration and high pressure homogenization process, maleimidated liposomes having hydrophobic ends (such as PLA, PLGA, PCL and DSPE) embedded in the lipid bilayer and PEG-maleimide ends located at surface of the lipid membranes can be obtained.

In addition, maleimide also can be directly introduced onto the surface of the drug loading system. For example, bifunctional linker (such as bifunctional propionic acid linker, wherein the active esters in the molecule can react with amino groups to generate maleimide groups), which can generate maleimide groups, is useful to introduce maleimide groups in the nanocarriers.

Another preferred coupling method is as follows:

Biotin is separately introduced into the targeting molecules and the drug loading system, and avidin is used as a bridging agent to construct targeted delivery system. In other words, avidin and biotinylated drug loading system are mixed firstly, and then the unbound sites bind with biotinylated targeting molecules.

For example, as to the preparation of biotinylated PEG-PLGA, PLGA-COOH (molecular weight is 20 kDa) is dissolved in dichloromethane. NHS and EDC in 8-fold amount of PLGA-COOH are added with stirring at room temperature to activate PLGA-COOH. The resultant active esters and NH₂-PEG-biotin (molecular weight is 3400 Da) are mixed and dissolved in chloroform. An appropriate amount of N,N-diisopropylethylamine is added, and the mixture reacts overnight. The unreacted PEG molecules are washed away with methanol, and the obtained product is precipitated with ether and dried in vacuo to provide PLGA-PEG-biotin. Then, a certain portion of PLGA-PEG-COOH and PLGA-PEG-biotin are mixed and dissolved in acetone, the mixture is slowly added dropwise into deionized water, and the acetone is removed by rotary evaporation at room temperature. The organic solvent is removed by ultrafiltration concentration, to obtain biotinylated PEG-PLGA nanoparticles. Biotinylated PEG-PLGA nanoparticles and avidin solution are incubated at room temperature for a certain time, and then the free avidin is removed by centrifuging and washing. A certain amount of biotinylated targeting molecules and the resultant product are stirred at room temperature, and then the free biotinylated targeting molecules are removed by centrifuging, thereby obtaining PEG-PLGA nanoparticles targeted drug delivery system.

Composition, the preparation method and use thereof

The composition of the present invention comprises the composite of the present invention and anti-tumor drugs carried in the nanocarrier of the composite. Based on total weight of the composition, the content of anti-tumor drugs usually is 1.5 to 3.0 wt %, preferably 2.0 to 3.0 wt %; and the content of targeting molecule is 1.11 to 22.2 wt %, and preferably 5.60 to 11.1 wt %.

In order to achieve better slow release and controlled release effect of anti-tumor drugs and to prevent the occurrence of in vivo opsonization, in the composition of the present invention, the particle size of the nanocarriers of the composite is preferably 200 nm or less, and preferably 10-200 nm.

The preferred anti-tumor drugs comprise (but are not limited to): doxorubicin, paclitaxel, docetaxel, cisplatin, mitoxantrone, daunorubicin, vincristine, all-trans retinoic acid, pharmorubicin, lurtotecan, irinotecan, 2-methoxyestradiol, gemcitabine, vinorelbine, 5-fluorouracil, methotrexate, capecitabine, lomustine, etoposide, or combinations thereof; and preferably comprise doxorubicin, paclitaxel, docetaxel, mitoxantrone, daunorubicin, irinotecan, gemcitabine, vinorelbine, capecitabine, or etoposide.

The preparation method of the composition in the present invention mainly comprises the steps of:

(1) providing a nanocarrier which is loaded with an anti-tumor drug;

(2) coupling the nanocarrier of step (1) with targeting molecules, thereby obtaining the composition.

In the invention, the nanocarrier can be prepared by ultrasonic emulsification. Preferably, the nanocarriers can be prepared by any of the following three methods:

(1) using a aqueous solution as a water phase in which hydrophilic anti-tumor drugs and hydrophilic membrane material (e.g., polyethylene glycol) are dissolved, and using an organic solvent (e.g. dichloromethane) as an oil phase in which oil-soluble emulsifier (e.g. sodium cholate) is dissolved; mixing the aqueous phase with the oil phase and stirring the mixture so as to achieve rough dispersion; emulsifying the mixture with ultrasonic cell crusher, thereby obtaining a water-in-oil type nanoemulsion; then under magnetic stirring, adding crosslinking agent (e.g. glutaraldehyde) into the resultant nanoemulsion to conduct cross-linking and cure; removing excess crosslinking agent and emulsifier, thereby obtaining biodegradable nanocarriers in which anti-tumor drugs are embedded;

(2) using an organic solvent (e.g. dichloromethane) as an oil phase in which hydrophobic anti-tumor drugs and hydrophobic membrane materials (e.g. PACA) are dissolved, and using a aqueous solution as a water phase in which water-soluble emulsifier (e.g. Pluronic F68, Dextran 70) is dissolved; mixing the oil phase with aqueous phase and stirring the mixture so as to achieve rough dispersion; emulsifying the mixture with ultrasonic cell crusher, thereby obtain an oil-in-water type nanoemulsion; then under magnetic stirring, adding crosslinking agent (e.g. glutaraldehyde) into the resultant nanoemulsion to conduct cross-linking and cure; removing excess crosslinking agent and emulsifier, thereby obtaining biodegradable nanocarriers in which anti-tumor drugs are embedded;

(3) using an aqueous solution as a water phase in which hydrophilic anti-tumor drugs are dissolved, and using an organic solvent as an oil phase in which hydrophobic membrane materials (e.g. PEG-PLA) and oil-soluble emulsifier (e.g. sodium cholate) are dissolve; mixing the aqueous phase with the oil phase and stirring the mixture so as to achieve rough dispersion; emulsifying the mixture with ultrasonic cell crusher, thereby obtaining a water-in-oil type nanoemulsion; and then adding the resultant water-in-oil type nanoemulsion into an aqueous phase in which water-soluble emulsifier dissolved and conducting ultrasonic emulsification, thereby obtaining a W/O/W type nanoemulsion; then under magnetic stirring, adding crosslinking agent (e.g. glutaraldehyde) into the resultant W/O/W type nanoemulsion to conduct cross-linking and cure; removing excess crosslinking agent and emulsifier, thereby obtaining biodegradable nanocarriers in which anti-tumor drugs are embedded.

The above nanocarriers of the present invention can also be prepared by using the solvent removal method. One preferred method includes the steps of: dissolving water-soluble antitumor drugs and oligopeptide or protein nanocarriers into NaCl aqueous solution, and adding ethanol dropwise with continuous magnetic stirring, and when the solution becomes a milky white suspension then adding glutaraldehyde for cross-linking and cure; removing excess crosslinking agent, thereby obtaining biodegradable nanoparticles in which anticancer drugs are embedded.

The reaction process of above step (2) is the same as that between nanocarriers and targeting molecules in the composite of the present invention.

It should be understood that the above composition may also be prepared by packaging the anti-tumor drugs into the prepared composite of the present invention.

The composition of the invention may be used to prepare anti-tumor medicaments, especially the medicaments for treating breast cancer, ovarian cancer, kidney cancer and stomach cancer.

Drugs, composition and administration method thereof

The drug or medicament of the present invention comprises an effective amount of the composite of the present invention, and a pharmaceutically acceptable carrier or excipient.

As used herein, the term “comprise” or “include” includes “comprising”, “substantially consisted of . . . ” and “consisted of . . . ”. As used herein, the term “pharmaceutically acceptable” component means that the component is suitable for human and/or animals without undue adverse side effects (such as toxicity, stimulation and allergy). In other words, the component is a kind of substances of reasonable benefit/risk ratio. As used herein, the term “effective amount” refers to an amount which can produce function or activity to human and/or animals and can be accepted by human and/or animals.

As used herein, the term “pharmaceutically acceptable carrier” refers to carriers used for the administration of the therapeutic agent, including various excipients and diluents. The term refers to such medicament carriers: they themselves are not essential active ingredients, and without undue toxic after application. Suitable carriers are well-known to those of ordinary skill in the art. The detailed discussion about the pharmaceutically acceptable excipient can be found in Remington's Pharmaceutical Sciences (Mack Pub. Co., N.J., 1991).

The formulation of the medicament of the present invention includes: solid preparation, liquid preparation or injection, and preferably injection.

The administration subject of the medicament in the present invention is a mammal, and preferably a human.

In another preferred embodiment of the present invention, the drugs or composition of the invention are administered one or more times per day, e.g. 1, 2, 3, 4, 5 or 6 times. The administration routes include, but are not limited to: oral administration, injection administration, intracavitary administration, transdermal administration; and preferably, injection administration includes intravenous, intramuscular, subcutaneous and intracavitary administration. When administering the drugs or composition of the present invention, in order to determine the specific dosage, the following factors should be taken into consideration: administration route, patient health status, etc., which are within the scope of the skills of skilled physician. The safe and effective amount of the composition of the present invention is usually at least about 10 mg or at least 85 mg/kg body weight per day, and in most cases no more than about 200 or no more than about 115 mg/kg body weight per day, and more preferably a dose of about 100 mg/kg body weight per day.

Compared with the prior art, the present invention has the following main advantages:

(1) the composite and composition of the present invention have good dispersion and stability in NaCl aqueous solution, PBS aqueous solution or serum, and there is no precipitation or agglomeration phenomenon;

(2) the composite and composition of the present invention can bind with breast cancer cells, ovarian cancer cells, kidney cancer cells and gastric cancer cells in high specificity, and have a strong targeting effect on tumor tissue;

(3) the composition and medicament of the present invention can directionally deliver anti-tumor drugs into the tumor cells, thereby effectively improving the drug concentration in the tumor cells, and have a strong killing effect against tumor cells, and have almost no toxic side effects on normal tissue and cells.

The features of the present invention mentioned above, or the characteristics mentioned in the examples can be arbitrarily combined. All the features disclosed in the specification of the present invention may be combined in any way, every feature disclosed in the specification can be replaced with any substituting feature which can provide identical, equal or similar purposes. Therefore, unless otherwise stated, the disclosed features are only general examples of equal or similar features.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacture's instructions. Unless indicated otherwise, parts and percentage are calculated by weight.

Unless otherwise defined, all professional and scientific terminology used in the text have the same meanings as known to the skilled in the art. In addition, any methods and materials similar or equal with the record content can apply to the methods of the invention. The method of the preferred embodiment described herein and the material are only for demonstration purposes.

Example 1 Preparation of composition [D-Arg²⁵]-NPY-ANP-TXT

(1) The preparation of BSA nanocarriers (ANP) embedded with TXT

10 mM NaCl aqueous solution of pH 10.8 was prepared, and the obtained solution was used to formulate BSA water solution having a concentration of 20 mg/mL. Then 2.0 mL ethanol was added into 2.0 mL BSA water solution, and after magnetic stirring for 10 min, 4.0 mL ethanol was added dropwise at a rate of 2.0 mL/min (the volume ratio of the amount of total added ethanol to the aqueous solution of the nanocarrier was 3.0), and the addition process was accompanied with continuous magnetic stirring. Once the dropwise addition of ethanol was finished, 8% glutaraldehyde aqueous solution was immediately added (the mass ratio of glutaraldehyde to BSA was 0.24) and cross-link (or cure) for 24 h. Then 1.0 mL glycine (40 mg/mL) was added to neutralize excess glutaraldehyde, and after reacting for 2.0 h, the sample was centrifuged (20,000 xg, 20 min), the resultant sample was washed twice with 10 mM NaCl aqueous solution, and finally after freeze-drying for 48 h, BSA nanocarriers were obtained. A solution of TXT was dispersed into an aqueous solution of 20 mg/mL BSA nanocarrier. Nanocarriers ANP-TXT having embedded anti-tumor drugs were prepared using the same method described above.

(2) Coupling targeting molecule [D-Arg²⁵]-NPY on surface of nanocarrier

Under the catalysis of EDAC (1-ethyl-(3-dimethylaminopropyl) carbodiimide) and via the chemical reaction between amino group of the targeting molecule [D-Arg²⁵]-NPY and carboxy group on surface of the nanocarrier ANP, the targeting molecule [D-Arg²⁵]-NPY was coupled onto surface of the nanocarrier. The specific preparation method was as follows: 500 μg/mL targeting molecule [D-Arg²⁵]-NPY solution was formulated by using phosphate buffer solution (PBS) as a solvent. 50 mg EDAC was dissolved into 10 mL targeting molecule solution (in ice bath), followed by addition of 90 mL ANP-TXT suspension (5.0 mg/mL) in PBS, and the mixture was magnetically stirred at room temperature, and the reaction lasted for 4-24 hrs. The sample was centrifuged (20,000 xg, 20 min), the resultant sample was washed twice with PBS. Finally, the sample was freeze-dried for 48 h to obtain the composition [D-Arg²⁵]-NPY-ANP-TXT which had a surface coupled with the targeting molecule and had anti-tumor drugs embedded in the nanocarrier.

The changes in particle size of composition [D-Arg²⁵]-NPY-ANP-TXT in NaCl solution, PBS solution and serum for 1-15 days are shown in Table 1 and FIG. 2.

TABLE 1

| | Mean diameter (nm) | | |
|---|---|---|---|
| Day(s) | NaCl | PBS | Serum |
| 1 | 116.6 | 115.2 | 118.6 |
| 3 | 115.2 | 116.8 | 117.5 |
| 5 | 113.4 | 114.3 | 116.4 |
| 7 | 111.6 | 116.9 | 115.2 |
| 9 | 111.8 | 115.2 | 111.9 |
| 11 | 112.6 | 113.4 | 114.6 |
| 13 | 110.5 | 111.6 | 113.1 |
| 15 | 110.4 | 110.3 | 111.9 |

As seen from Table 1 and FIG. 2, the composite nanoparticles had uniform particle size and good dispersion in three different solvents, and the particle size was stabilized between 110˜120 nm.

Measured by a method using nanoparticle size analyzer, each of the composite nanoparticles (compositions) had a PDI index of less than 0.5.

Example 2 Activity test of composition [D-Arg²⁵]-NPY-ANP-TXT on tumor cells MCF-7 and HEC-1B-Y5

(1) MTT test (cell toxicity test)

1. formulating a single cell suspension with a medium containing 10% fetal calf serum, and then seeding the suspension into 96-well plates in 1.0×10⁵ cells per well. The volume of each well was 150 μL.

2. placing the plates into a cell incubator of 37° C. and culturing for 24 h.

3. absorbing and discarding the supernatant of the medium in the well, and adding 2004, fresh medium containing TXT or 2000 μL, solution containing composition [D-Arg²⁵]-NPY-ANP-TXT with same concentration of TXT.

4. placing into a cell incubator of 37° C. and culturing for 4 h, absorbing and discarding the supernatant of the medium in the well, and then replacing with fresh medium contain no medication or nanoparticle composition, and continuing to culture for 44 h.

5. adding 10 μL MTT solution (5 mg/ml, in PBS, pH=7.4) into each well, placing the plates into a cell incubator of 37° C. and continuing to culture for 4 h, terminating the culture, and then absorbing and discarding the supernatant of the medium in the well.

6. adding 150 μL DMSO into each well, oscillating for 10 min, so that the crystal was fully thawed.

7. selecting 550 nm wavelength, and measuring the light absorbance for each well with enzyme-linked immunosorbent monitor and recording the results. The test results are shown in Table 2.

The cells used in cell experiments comprised human breast cancer MCF-7 cells, human endometrial tumors HEC-1B-Y5 cells (Purchased from American Type Culture Collection (ATCC) and Sciencell company(USA))

TABLE 2
Killing effect of composition [D-Arg²⁵]-NPY-
ANP-TXT against MCF-7 and HEC-1B-Y5

| | survival rate of cell (%) | | | |
|---|---|---|---|---|
| | | | composition | composition |
| | | | [D-Arg²⁵]-NPY- | [D-Arg²⁵]-NPY- |
| | TXT | TXT | ANP-TXT | ANP-TXT |
| C_{TXT} | Tumor cells | | Tumor cells | |
| (μg/mL) | MCF-7 | HEC-1B-Y₅ | MCF-7 | HEC-1B-Y₅ |
| 0.0 | 100 | 100 | 100 | 100 |
| 0.5 | 90 | 91 | 93 | 98 |
| 1.0 | 80 | 79 | 86 | 90 |
| 2.0 | 50 | 55 | 60 | 85 |
| 5.0 | 20 | 40 | 32 | 75 |
| 10.0 | 20 | 30 | 25 | 60 |

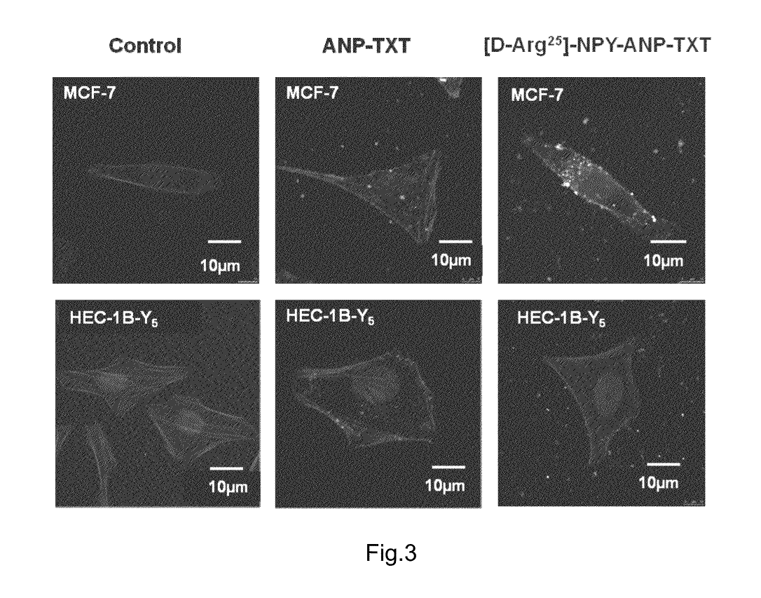

It can be seen from Table 2 and FIG. 3 that docetaxel (TXT) has excellent killing effect against MCF-7 cells and HEC-1B-Y5 cells, and the composition [D-Arg²⁵]-NPY-ANP-TXT has an excellent killing effect against MCF-7 cells, however, while the killing effect of the composition against HEC-1B-Y5 cell is not significant. Therefore, these results indicate that the composition [D-Arg²⁵]-NPY-ANP-TXT can unexpectedly and significantly kill MCF-7 cells in highly selective, and has an excellent killing effect.

Example 3 Activity test of composition [D-Arg²⁵]-NPY-ANP-TXT to different tumor cells

(1) The activity test method was the same as that in Example 2. The test results are shown in Tables 3-1 and 3-2. In this example, the cells used in cell experiments comprised human ovarian tumor UWB 1.289 cells, human gastric cancer GIST-H1 cells, human kidney tumors SW-13 cells, human brain tumor SMS-KAN cells. Normal cells comprised human breast epithelial cells MCF-10a, human ovarian surface epithelial cells HOSEpiC, human renal cortical epithelial cells HRCEpiC, human gastric cell GES-1, human brain astrocytes HA, human endometrial epithelial cells HUM-CELL-0111. (Purchased from American Type Culture Collection (ATCC), Sciencell company (USA) and the cell bank of Chinese Science Academy Type Culture Collection Committee)

TABLE 3-1
Comparison of killing effects of composition
[D-Arg²⁵]-NPY-ANP-TXT against different tumor cells

| | survival rate of cell (%) | | | | | |
|---|---|---|---|---|---|---|
| | tumor cells | | | | | |
| C_{TXT} | | | | | SMS- | HEC- |
| (μg/mL) | MCF-7 | UWB1.289 | SW-13 | GIST-H1 | KAN | 1B-Y5 |
| 0.0 | 100 | 100 | 100 | 100 | 100 | 100 |
| 0.5 | 93 | 94 | 90 | 91 | 97 | 95 |
| 1.0 | 86 | 88 | 85 | 82 | 89 | 86 |
| 2.0 | 60 | 65 | 63 | 60 | 82 | 80 |
| 5.0 | 32 | 40 | 35 | 33 | 76 | 74 |
| 10.0 | 25 | 26 | 20 | 24 | 71 | 70 |

TABLE 3-2
Comparison of killing effects of composition
[D-Arg²⁵]-NPY-ANP-TXT against normal cells

| | survival rate of cell (%) | | | | | |
|---|---|---|---|---|---|---|
| | normal cells | | | | | |
| C_{TXT} | | | | GES- | | HUM- |
| (μg/mL) | MCF-10a | HOSEpiC | HRCEpiC | 1 | HA | CELL-0111 |
| 0.0 | 100 | 100 | 100 | 100 | 100 | 100 |
| 0.5 | 93 | 94 | 90 | 91 | 97 | 98 |
| 1.0 | 90 | 91 | 88 | 90 | 94 | 95 |
| 2.0 | 88 | 84 | 81 | 84 | 87 | 85 |
| 5.0 | 85 | 78 | 78 | 80 | 82 | 81 |
| 10.0 | 78 | 75 | 76 | 75 | 78 | 75 |

It can be seen from Table 3-1 that the composition [D-Arg²⁵]-NPY-ANP-TXT of the present invention has excellent killing effect against UWB1.289 cells, SW-13 cells and GIST-H1 cells as against MCF-7 cells. However, the killing effect of the composition against SMS-KAN cell is not significant. Therefore, these results indicate that the composition [D-Arg²⁵]-NPY-ANP-TXT can kill UWB1.289 cells, SW-13 cells and GIST-H1 cells in high selectivity, and has an excellent killing effect.

It can be seen from the test results of Table 2, Table 3-1 and Table 3-2 that the composition [D-Arg²⁵]-NPY-ANP-TXT can bind with breast cancer, ovarian cancer, renal cancer and gastric cancer cells in high specificity, and it has strong targeting effect against tumor cells and can directionally deliver anti-tumor drugs into the tumor cells, thereby effectively improving the drug concentration in tumor cells, and has a strong killing effect against tumor cells and has almost no toxic side effects to normal tissues and cells.

Example 4 Activity test of different docetaxel-embedded compositions on MCF-7 and UWB1.289 cells

(1) Preparation of the composition

(a) Preparation of TXT-embedded chitosan nanocarriers composition

(i) Preparation of TXT-embedded chitosan nanocarriers

0.2% (w/v) chitosan solution was formulated and the solvent was 1% (w/v) acetic acid. The docetaxel (TXT) was dispersed into the chitosan solution, and pH of the solution was adjusted to 4.7-4.8 with sodium hydroxide. 0.3% (w/v) sodium tripolyphosphate (TPP) water solution was formulated. Under magnetic stirring, 0.1 mL TPP solution was added into 0.5 mL the chitosan solution, thereby obtaining ion-crosslinked TXT-embedded chitosan nanocarriers.

(ii) coupling the targeting molecule onto surface of chitosan nanocarriers

The targeting molecule coupling reaction was carried out on the surface of the resultant nanocarriers in the same way as step (2) in Example 1 except that the targeting molecule [D-Arg²⁵]-NPY was replaced with those listed in Table 5.

(b) Preparation of TXT-embedded BSA nanocarriers (ANP) composition

The preparation was conducted by referring to the procedure of Example 1 and replacing the targeting molecule [D-Arg²⁵]-NPY with those listed in Table 5.

(2) Cytotoxicity test

The activity tests were performed by referring to the method in Example 2, wherein the cells used in experiments comprised MCF-7 cells and UWB 1.289 cells.

The test results are shown in Table 5, wherein

“+” means that the composition has killing effect against tumor cells,

“−” represents that the composition has almost no killing effect against tumor cells or the killing effect is weak. Specific meanings of symbols are shown in Table 4 (taking one representative concentration value, and taking different symbols according to the range of values):

TABLE 4

| C_{TXT} | | |
|---|---|---|
| (μg/mL) | value(s) | symbols |
| 5 | >80 | −−− |
| | 60 < the survival rate of cell < 80 | −− |
| | 50 < the survival rate of cell < 60 | − |
| | 40 < the survival rate of cell < 50 | + |
| | 30 < the survival rate of cell < 40 | ++ |
| | 20 < the survival rate of cell < 30 | +++ |

TABLE 5
Comparison of killing effects of different compositions
against MCF-7 and UWB1.289 cells

| | Nanocarrier | | | |
|---|---|---|---|---|
| | BSA nanocarrier | | chitosan nanocarrier | |
| Target molecule | MCF-7 | UWB1.289 | MCF-7 | UWB1.289 |
| [D-Arg²⁵]-NPY | ++ | ++ | ++ | ++ |
| [D-His²⁶]-NPY | ++ | ++ | ++ | ++ |
| [D-Arg²⁵, D-His²⁶]-NPY | ++ | ++ | ++ | ++ |
| [D-His²⁶, Pro³⁴]NPY | ++ | ++ | ++ | ++ |
| [Phe⁷, Pro³⁴]pNPY | +++ | +++ | +++ | +++ |
| [Arg⁶, Pro³⁴]pNPY | +++ | +++ | +++ | +++ |
| [Asn⁶, Pro³⁴]pNPY | +++ | +++ | +++ | +++ |
| [Cys⁶, Pro³⁴]pNPY | +++ | +++ | +++ | +++ |
| [Phe⁶, Pro³⁴]pNPY | +++ | +++ | +++ | +++ |
| [Pro³⁰, Nle³¹, Bpa³², | +++ | +++ | +++ | +++ |
| Leu³⁴]NPY(28-36) | | | | |
| [Pro³⁰, Nal³², | +++ | +++ | +++ | +++ |
| Leu³⁴]NPY(28-36) | | | | |
| [Pro³⁰, Nle³¹, Nal³², | +++ | +++ | +++ | +++ |
| Leu³⁴]NPY(28-36) | | | | |
| BIBO3304 | +++ | +++ | +++ | +++ |
| PD160170 | ++ | ++ | ++ | ++ |
| LY366258 | + | ++ | + | ++ |
| J-104870 | ++ | ++ | ++ | ++ |
| LY 357897 | + | + | + | + |
| J-115814 | ++ | ++ | ++ | ++ |

Example 5 Activity test of different docetaxel-embedded compositions on GIST-H1 and SW-13 cells

The preparation method and cytotoxicity tests of different compositions were conducted by referring to the procedure in Example 4. The test results are shown in Table 6.

TABLE 6
Comparison of killing effects of different compositions
against GIST-H1 and SW-13 cells

| | Nanocarrier | | | |
|---|---|---|---|---|
| | BSA nanocarrier | | chitosan nanocarrier | |
| Target molecule | SW-13 | GIST-H1 | SW-13 | GIST-H1 |
| [D-Arg²⁵]-NPY | ++ | ++ | ++ | ++ |
| [D-His²⁶]-NPY | ++ | ++ | ++ | ++ |
| [D-Arg²⁵, D-His²⁶]-NPY | ++ | ++ | ++ | ++ |
| [D-His²⁶, Pro³⁴]NPY | ++ | ++ | ++ | ++ |
| [Phe⁷, Pro³⁴]pNPY | +++ | +++ | +++ | +++ |
| [Arg⁶, Pro³⁴]pNPY | +++ | +++ | +++ | +++ |
| [Asn⁶, Pro³⁴]pNPY | +++ | +++ | +++ | +++ |
| [Cys⁶, Pro³⁴]pNPY | +++ | +++ | +++ | +++ |
| [Phe⁶, Pro³⁴]pNPY | +++ | +++ | +++ | +++ |
| [Pro³⁰, Nle³¹, Bpa³², | +++ | +++ | +++ | +++ |
| Leu³⁴]NPY(28-36) | | | | |
| [Pro³⁰, Nal³², | +++ | +++ | +++ | +++ |
| Leu³⁴]NPY(28-36) | | | | |
| [Pro³⁰, Nle³¹, Nal³², | +++ | +++ | +++ | +++ |
| Leu³⁴]NPY(28-36) | | | | |
| BIBO3304 | +++ | +++ | +++ | +++ |
| PD160170 | ++ | ++ | ++ | ++ |
| LY366258 | + | ++ | + | ++ |
| J-104870 | ++ | ++ | ++ | ++ |
| LY 357897 | + | + | + | + |
| J-115814 | ++ | ++ | ++ | ++ |

Example 6 Activity test of different docetaxel-embedded compositions on SMS-KAN and HEC-1B-Y5 cells

The preparation method and cytotoxicity tests of different compositions were conducted by referring to the procedure in Example 4. The test results are shown in Table 7.

TABLE 7
Comparison of killing effects of different compositions
against SMS-KAN and HEC-1B-Y5 cells

| | Nanocarrier | | | |
|---|---|---|---|---|
| | BSA nanocarrier | | chitosan nanocarrier | |
| | | HEC- | | HEC- |
| Target molecule | SMS-KAN | 1B-Y5 | SMS-KAN | 1B-Y5 |
| [D-Arg²⁵]-NPY | −− | −− | −− | −− |
| [D-His²⁶]-NPY | −− | −− | −− | −− |
| [D-Arg²⁵, D-His²⁶]-NPY | −− | −− | −− | −− |
| [D-His²⁶, Pro³⁴]NPY | −− | −− | −− | −− |
| [Phe⁷, Pro³⁴]pNPY | −−− | −−− | −−− | −−− |
| [Arg⁶, Pro³⁴]pNPY | −−− | −−− | −−− | −−− |
| [Asn⁶, Pro³⁴]pNPY | −−− | −−− | −−− | −−− |
| [Cys⁶, Pro³⁴]pNPY | −−− | −−− | −−− | −−− |
| [Phe⁶, Pro³⁴]pNPY | −−− | −−− | −−− | −−− |
| [Pro³⁰, Nle³¹, Bpa³², | −−− | −−− | −−− | −−− |
| Leu³⁴]NPY(28-36) | | | | |
| [Pro³⁰, Nal³², | −−− | −−− | −−− | −−− |
| Leu³⁴]NPY(28-36) | | | | |
| [Pro³⁰, Nle³¹, Nal³², | −−− | −−− | −−− | −−− |
| Leu³⁴]NPY(28-36) | | | | |
| BIBO3304 | −−− | −−− | −−− | −−− |
| PD160170 | −− | −− | −− | −− |
| LY366258 | −− | −− | −− | −− |
| J-104870 | −−− | −−− | −−− | −−− |
| LY 357897 | −− | −− | −− | −− |
| J-115814 | −−− | −−− | −−− | −−− |

It can be seen from the test results of Tables 5-7 that the compositions coupled with the targeting molecule of the present invention have strong killing effect against MCF-7, UWB 1.289, GIST-H1 and SW-13 cells. When C_{TXT} was 5 μg/mL, the survival rates of MCF-7, GIST-H1 and SW-13 cells were substantially below 30%, and the survival rate of UWB 1.289 cell was 60%. However, the killing effects against SMS-KAN and HEC-1B-Y5 cells were not significant, the survival rates of cell were substantially above 80%. Therefore, the compositions of the present invention have good selectivity, have strong targeting effect against breast cancer, ovarian cancer, renal cancer and stomach cancer cells, and have strong killing effect against tumor cells and little killing effect against brain tumors and endometrial tumor cells.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A composite, wherein the composite comprises:
a nanocarrier; and
a targeting molecule coupled to a surface of the nanocarrier;
wherein the targeting molecule is selected from the group consisting of: [D-Arg²⁵]-NPY, [D-His²⁶]-NPY, [D-Arg²⁵, D-His²⁶]-NPY, [Arg⁶, Pro³⁴]pNPY, [Asn⁶, Pro³⁴]pNPY, [Cys⁶, Pro³⁴]pNPY, [Phe⁶, Pro³⁴]pNPY, [Arg⁷, Pro³⁴]pNPY, [D-His²⁶, Pro³⁴]NPY, [Phe⁷, Pro³⁴]pNPY, [Pro³⁰, Nle³¹, Bpa³², Leu³⁴]NPY(28-36), [Pro³⁰, Nal³², Leu³⁴]NPY(28-36), [Pro³⁰, Nle³¹, Nal³², Leu³⁴]NPY(28-36), BIBO3304, PD160170, LY366258, J-104870, LY 357897, J-115814, and combinations thereof;
and the nanocarrier has a particle size which is 200 nm or less and a polydispersity index (PDI) which is smaller than 0.5.

2. The composite according to claim 1, wherein the content of targeting molecule is 1.11 to 22.2 wt % of total weight of the composite.

3. The composite according to claim 1, wherein the composite has one or more of the following characteristics:
(a) the composite binds with breast cancer cells, ovarian cancer cells, renal cancer cells or stomach cancer cells in high specificity;
(b) the particle size of the nanocarrier is 10-200 nm.

4. The composite according to claim 1, wherein the nanocarrier is selected from the group consisting of: protein-based nanoparticle, oligopeptide-based nanoparticle, phospholipid-based nanoliposome, polysaccharide-based nanoparticle, polyether-based nanoparticle, polyester-based nanoparticle, and polyester-based polymeric micelle.

5. A composition, wherein the composition comprises:
a composite of claim 1; and
an anti-tumor drug carried in the nanocarrier of the composite.

6. A preparation method of the composition of claim 5, which comprises the following steps:
(1) providing a nanocarrier loaded with an anti-tumor drug; and
(2) coupling the nanocarrier of step (1) with a targeting molecule, thereby obtaining the composition.

7. The preparation method of claim 6, wherein the coupling reaction is selected from:
(1) a condensation reaction of a carboxyl group and an amino group;
(2) an addition reaction of a sulfhydryl group and an maleimide group; or
(3) a non-covalent binding of avidin and biotin.

8. A use of the composite of claim 1 for preparation of a medicament for treatment of cancer.

9. A use of the composition of claim 5 for preparation of a medicament for treatment of cancer.

10. A medicament which comprises:
the composite of claim 1;
an anti-tumor drug carried in the nanocarrier of the composite; and
a pharmaceutically acceptable carrier.
